(19) United States
(12) Patent Application Publication
(10) Pub. No.: US 2007/0150008 A1
(43) Pub. Date: Jun. 28, 2007

(54) Defibrillation electrode having drug delivery capablity

(75) Inventors: Janice L. Jones, Clarksbury, MD (US); David E. Snyder, Bainbridge Island, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: Koninklijke Philips Electronics, N.V., Eindhoven (NL)

(21) Appl. No.: 10/599,112

(22) PCT Filed: Mar. 16, 2005
(86) PCT No.: PCT/IB05/50927

§ 371 Date: Sep. 20, 2006

Related U.S. Application Data
(60) Provisional application No. 60/556,220, filed on Mar. 25, 2004.

(51) Int. Cl. A61N 1/39

(52) U.S. Cl. 607/3; 607/5; 604/20

(57) ABSTRACT

A defibrillation electrode includes a conductive member having first and second opposite side surfaces, a non-conductive backing connected to the first surface of the conductive member, and at least one drug delivery medium in electrical communication with the second surface of the conductive member. The drug delivery medium is adapted to be in surface contact with a patient so as to impart transdermal drug delivery when the electrode is in communication with a power supply.

The present invention relates generally to electrotherapy devices of the type known as “external defibrillators.” More specifically, the present invention relates to an external defibrillator having patch electrodes which create an electrical pathway for delivering a defibrillation shock and facilitate the delivery of drugs into the patient's bloodstream without the use of needles.

Resuscitation from sudden cardiac arrest (SCA) often requires the use of various pharmaceutical agents, such as epinephrine and lidocaine, in order to improve perfusion and contractile state, stimulate spontaneous contraction and regulate dysrhythmias. Current research also suggests that pre- and/or post-defibrillation drug “cocktails” may help protect the cardiac cells from ischemia and reperfusion related injury. Unfortunately, these techniques currently require intravenous or endotracheal access, and are limited to use by advanced life support practitioners.

The transdermal applications of drugs is well established, including over-the-counter products for the suppression of smoking urges (known as the “nicotine patch”) and the treatment of seasickness. Transdermal patches offer a method of drug administration which is easily mastered by people without medical training. Unfortunately, the skin's poor permeability prevents the timely delivery of most drugs at therapeutic levels that would be useful for emergency resuscitation.

It is well known that the transdermal delivery of ionized drugs can be accelerated several hundred percent via iontophoresis, which is the application of a small electric potential (typically less than 30 volts) across the medicated patch/skin barrier. Recently, research has been done with pulses of higher voltage (30 to several hundred volts with a duration of one to several hundred milliseconds) in a process known as electroporation. In electroporation, the higher voltage pulses establish large aqueous pathways for the transfer of macromolecules at therapeutically relevant rates, demonstrating a drug flux enhancement of up to four orders of magnitude. Electroporation may in turn be enhanced by the subsequent application of iontophoretic level voltages. Unfortunately, electrically enhanced transdermal delivery of drugs requires the use of specialized electrical equipment in addition to the medicated patches.

A class of portable, external defibrillators has evolved from the recognition that laypersons or lightly to moderately trained personnel are at times the first to administer potentially lifesaving first aid. One such defibrillator is described in U.S. Pat. No. 5,607,454 (“the '454 patent”), assigned to Heartstream, Inc., in which a defibrillator weights a total of less than four pounds and has a volume of less than 150 cubic inches. This electrotherapy device includes a power source and two electrodes that make electrical contact with the patient. A premium is placed on making the device as simple as possible to facilitate rapid operation while minimizing the risk of accidental shock.

Preferably, the electrodes used in devices of the type shown in the '454 patent are quickly and easily positioned and attached to the patient. Several particularly advantageous electrode structures for accomplishing these goals have been developed, such as those shown in U.S. Pat. No. 5,466,244 (“the '244 patent”), assigned to Heartstream, Inc. FIG. 1 of the present disclosure illustrates a portable defibrillator 10 with two electrodes 12 and 14 properly positioned and attached to a patient. The electrodes of the type shown in the present disclosure include a flexible substrate 16 which is made of polymeric, non-conductive material such as polyester. An electrically conductive metallic foil 18, made of a suitable material such as tin, is located on one surface of the substrate 16, and is electrically connected to control circuitry of the defibrillator 10. An electrically conductive gel layer 20 has an adhesive property that permits direct connection to the patient without having to separately tape or otherwise secure the electrodes to the patient. A protective covering (not shown) is typically provided over the patient-contacting surface of the gel layer 20 to prevent drying out and to facilitate storage.

A need exits to make pharmaceutical intervention available in a more accessible manner, by use of machine automation that in turn makes important treatment available to less trained rescuers and, consequently, a broader population of SCA victims.

The present invention is directed to a defibrillator with systems for performing the electrically enhanced transdermal delivery of drugs. The delivery system includes electrically connected medication patches which may be separate from, or incorporated into, the defibrillation electrodes.

The electrical connection to the medicated patch may be separate from, or coincident with, the defibrillation patch. The defibrillation patches may be used to apply an electric potential across the medicated patch in either a multi-patch electrode or a separate electrode. The defibrillator may also synchronize electrical pulses for the enhancement of drug delivery to features of the patient's ECG so as to minimize the possibility of electrically inducing a cardiac arrhythmia. In one particular embodiment of the invention, the defibrillator may incorporate an algorithm which makes use of a patient-dependent parameter such as characteristics of the ECG, to provide guidance to a rescuer, or to automate the administration of drugs via electrical activation of the medicated patch.

One aspect of the invention is to provide an apparatus that provides the dual functions of providing defibrillation and drug delivery. The apparatus includes a power source, at least one defibrillator electrode connectable to a subject and being electrically coupled to the power source to receive electric energy sufficient to defibrillate the subject, and a drug delivery electrode connectable to the subject and being electrically coupled to the power source to received electric energy sufficient to deliver a drug to the subject.

In another aspect of the invention, a therapeutic agent, or drug, is incorporated into the gel layer that is typically used to attach a defibrillation electrode to the subject. Thus, a conventional defibrillation electrode of the type that has a conductive layer or metal foil and a gel layer covering the conductive layer is modified by dispersing a therapeutic agent into the gel layer. When the drug is incorporated into the gel layer the circuitry, power supply and/or programming of the base unit can be modified so that a drug delivery voltage, or electric energy, is applied to the electrode before, during and/or after application of the defibrillation voltage or electrical energy is applied. Such modifications can be hard wired into the control circuitry, or can be programmed into a microprocessor, controller or other suitable processing means.

In the disclosed embodiments the control circuit is constructed to minimize user intervention so that, for example, the operator can simply attach the electrodes to the subject and switch on the defibrillator. Operating procedures can be simplified according to any of the control and operation procedures of any known variety.

A further variation of the invention involves use of a single electrode structure to carry electrically isolated regions, each being supplied with a different electric energy level, such that the higher energy level is applied to the defibrillation region and the lower energy level is applied to the drug delivery region. This embodiment requires coupling each to a different source of energy, or to a different power distribution circuit. For example, to impart the different energy levels, the apparatus may include a primary power supply for supplying defibrillation energy to the defibrillation electrodes and secondary power supply for supplying drug delivery energy to the drug delivery electrode. The secondary supply may be coupled between one of the defibrillation electrodes and the drug delivery electrode.

Further aspects of the invention will become more apparent from the following detailed description when taken in conjunction with the illustrative embodiments in the accompanying drawings.

### IN THE DRAWINGS

FIG. 1 is a schematic view of a defibrillation apparatus known in the art;

FIG. 2 is an enlarged, partial cross-sectional view of one of the electrodes shown in FIG. 1, taken along line 11-11;

FIG. 3 is a cross-sectional view similar to FIG. 2, showing an embodiment of the invention in which an electrode has a conductor having a defibrillation portion electrically isolated from a drug delivery portion;

FIG. 4 is a top view showing a defibrillation electrode according to another embodiment of the invention, in which drug delivery sections are provided with separate leads for coupling separately to a power source;

FIG. 5 is a schematic view of a defibrillation apparatus according to the present invention showing two defibrillation electrodes, either of which could be used to carry a therapeutic agent in its gel layer, or in separate, electrically isolated regions of the gel layer;

FIG. 6 is a schematic view of the circuitry for the apparatus of FIG. 5;

FIG. 7 is a schematic view of a defibrillation apparatus according to another embodiment of the invention in which a separate drug delivery electrode is provided;

FIG. 8 is a schematic view of the circuitry for the apparatus of FIG. 7; and

FIG. 9 is a flow diagram showing the process for operating the apparatus.

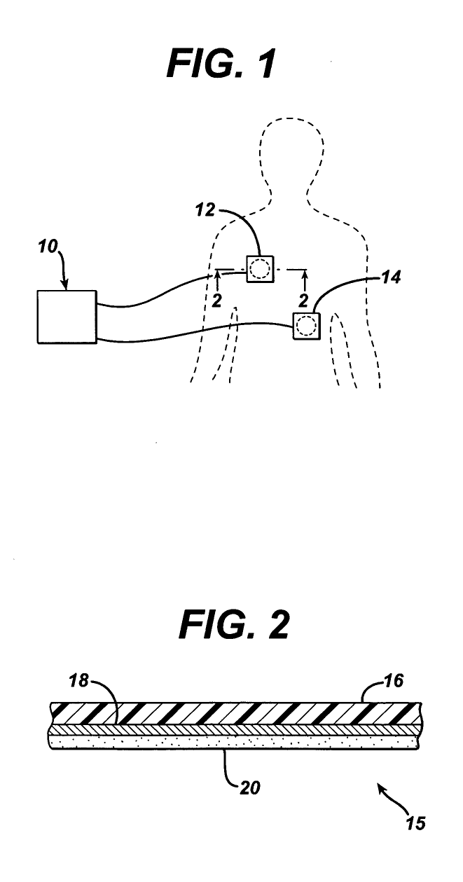

The present invention combines a defibrillator electrode incorporating or used in conjunction with a transdermal drug delivery system. Drug delivery can be enhanced using electro-motive forces which can be established and controlled by the control circuitry of the defibrillator. Electro-motive enhancements include, but are not limited to, electro-osmosis and iontophoresis. Preconditioning includes, but is not limited to, electroporation. An advantage to the present invention is that the existing electrode structures need little modification to be adapted for drug delivery.

An example can be illustrated with reference to FIG. 2, which has been used to illustrate the prior art electrodes. The gel layer 20 can be modified to include an active therapeutic agent within the gel material. In such applications, the structure would not appear physically different from the prior art, although the gel layer would be modified to include the active therapeutic agent.

Thus, a defibrillation electrode 15 according to the present invention is configured for attachment to a subject, such as someone undergoing a cardiac event. The electrode includes a flexible substrate 16 and a conductive member 18 having an outer surface that would face the subject. The conductive member 18 could be a metal foil, as is used in some prior art devices. A gel layer 20 covers at least a portion of the outer surface of the conductor 18, as in prior art devices, to aid in attaching the electrode to the skin of the subject and establishing a good electrical contact. The gel 20 includes a therapeutic agent dispersed within at least a portion thereof in an amount sufficient to establish a desired dosage. The therapeutic agent transports to the subject under the influence of an electromotive force applied through the conductive member.

The defibrillator circuitry is programmed to operate in an additional mode, called the “electro-motive” mode, in which an electric potential can be established between the electrodes that causes the active agent to migrate from the gel into the bloodstream of the patient through the skin. Iontophoresis provides an electrical driving force to move charged molecules into the subject's skin and thus into the bloodstream. Electroporation, which may also be a desired electro-motive force, involves application of electric field pulses that create transient aqueous pathways in lipid bilayer membranes, causing a temporary alteration of skin structure. The actual transport of charged molecules during pulsing occurs predominantly by electro-osmosis and iontophoresis.

The precise voltages, pulse rates, and nature of the electric field (a.c. vs. DC) can be selected depending on the type of active agent being administered, as well as the dosages. An a.c. voltage will generally not be desirable as an electromotive force but could be used for electroporation.

In keeping with the general goal of providing a defibrillator that is easily operated by the unskilled or layperson, the control circuitry can provide a defibrillation voltage to the electrode 15 as well as a drug delivery voltage. Preferably, the control unit or base unit includes simple operation switches so that the drug delivery function is provided automatically, such as by applying the drug delivery voltage for predetermined times and durations, such as before, during and/or after application of the defibrillation voltage.

A microprocessor or microcontroller within the control circuitry is programmed to automatically perform electroporation, electromotive drug delivery and/or defibrillation in a pre-determined sequence. The sequence of these therapies may also be adapted to a particular patient according to a patient-dependent parameter. The voltages and/or current necessary to perform both drug delivery and defibrillation shock can be predetermined or can be selected by the microprocessor in a look-up table, once the type of drug is determined either by an automated algorithm or by manual selection. A user can manually select a drug type by dial, push-button or by other suitable means.

The types of drugs to be administered can be a variety of cardiac drugs, and virtually any pharmaceutically active agent that might be indicated for treatment of ventricular fibrillation. One example of a cardiac drug is a heart stimulant such as epinephrine. Epinephrine is an endogenous catecholamine with potent α- and β-adrenergic stimulating properties. In cardiac arrest, α-adrenergic-mediated vasoconstriction is the most important pharmacologic action because restoration of aortic diastolic pressure is a critical determinant of success or failure of resuscitation. Vasoconstriction elevates perfusion pressure, thus enhancing delivery of oxygen to the heart. Other cardiac drugs that could be delivered using the present invention include adenosine, bretylium, atropine sulfate, and lidocaine. Lidocaine is used to suppress ventricular ectopy and to raise the threshold for ventricular fibrillation.

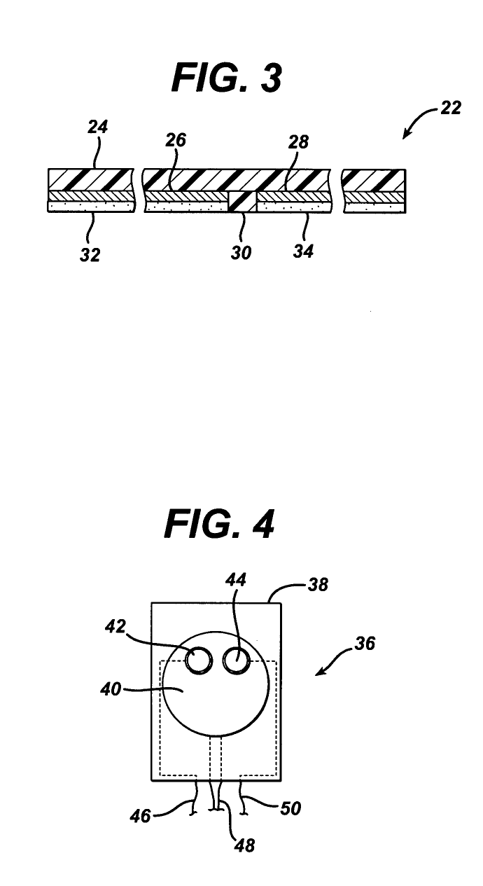

FIG. 3 illustrates an alternative embodiment of a defibrillation electrode 22 which is attachable to a subject as in the previous embodiment. An electrically non-conductive substrate 24 has opposite surfaces, one of which is connected to a first conductive member 26 having an outer surface, and a second conductive member 28 having an outer surface. The first and second conductive members 26 and 28 are electrically isolated from each other, or substantially isolated from each other, by insulator 30.

A first gel layer 32 is connected to at least a portion of the outer surface of the first conductive member 26, and a second gel layer 34 is connected to at least a portion of the second conductive member 28. As illustrated, the insulator 30 also electrically isolates the first gel layer 32 from the second gel layer 34, although an air gap may also provide sufficient isolation. In this embodiment the therapeutic agent is dispersed within at least a portion of the second gel layer 34, so that the therapeutic agent transports to the subject under the influence of an electromotive force applied through the second conductive member 28.

The electrical isolation provided herein allows for the power source, or multiple power sources, to provide electric energy to the different conductive members at different levels, at different times, and for different purposes. Thus, conductive member 26 could be connected to a first power source, and conductive member 28 connected to a second, different power source. Alternatively, they could be connected through different circuitry and/or switch combinations to provide different levels of energy from the same power source at the same or at different times.

The second gel layer 34 may consist of areas containing different drugs and/or additional doses of a drug. Optionally, different defibrillation electrodes can be provided with different drugs and different doses of drugs, and may thus be preconnected to a particular defibrillation device or may be connectable to the device with instructions as to which of the different drug-carrying electrodes to use. It is recognized, however, that in most cases user intervention is to be simplified, so that preferred embodiments would require no user selection of electrodes.

According to another embodiment of the present invention multiple therapeutic drug “patches” can be provided on a single electrode, for the purpose of providing additional dosage of a single drug, or for simultaneously administering two drugs. Referring to FIG. 4, a defibrillation electrode 36 has a non-conductive substrate 38 which carriers three different conductors: a first one corresponding to the larger diameter circle, and second and third ones corresponding to the smaller diameter circles. Each conductor is electrically isolated from the other. A first gel layer 40 covers the first conductor while gel layers 42 and 44 cover the second and third conductors, respectively. The area around each of the gel layers 42 and 44 represents insulator material or a gap which electrically insulates the gel layers 42 and 44 from the gel layer 40.

As shown in FIG. 4, each of the conductors is connected to a separate electrical lead, such as leads 46, 48, and 50, so that a different and separate amount of electric energy can be applied to each. For example, a defibrillation voltage can be applied to the first electrode, while no voltages are applied to the second and third electrodes, and drug delivery voltages can be applied to the second and third electrodes while no voltage is applied to the first electrode. Timing, sequence, duration and levels of applied electric energy can be determined by the control circuits of the defibrillator.

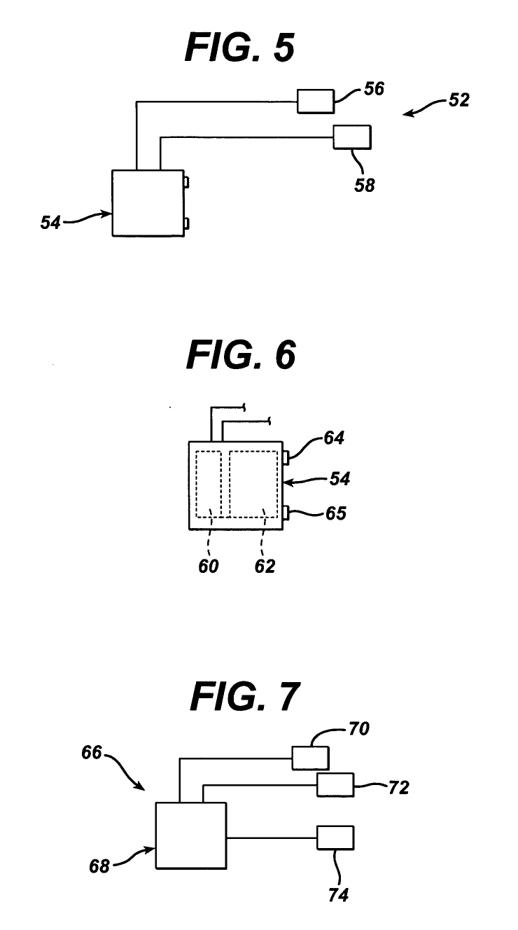

Referring to FIG. 5, a defibrillation apparatus 52 includes a base unit 54 and a pair of defibrillation electrodes 56 and 58. In most respects, the apparatus 52 corresponds to a type of device known as automated external defibrillators (“AED's”), which are highly portable and designed to be used by laypersons or otherwise by those who are unskilled in the medical arts. Operation is automated to the greatest extent possible, so that the operator can simply attach the electrodes and turn the device on and most every other function that follows is performed automatically by automated diagnosis and/or pre-programming.

The base unit 54 includes a power supply (not shown in FIG. 5) and a control circuit which makes delivery of a defibrillation shock to a subject via the electrodes 56 and 58. The electrodes are easily attached to the subject's skin prior to initiation of the defibrillation shock. The power supply and control circuitry for establishing a defibrillation shock are known and described in other patents assigned to Heartstream, Inc.

In order to induce drug delivery through the defibrillation electrodes one of the electrodes 56 or 58 is provided with a therapeutic agent in the gel layer so that, when an appropriate electro-motive force is applied, the therapeutic agent transports across the skin from the gel layer and into the bloodstream of the subject.

As noted above, the electrodes may carry the therapeutic agent on the same electric circuit, or on electrically isolated circuits, and preferably the latter. Isolated circuits will allow the administration of a drug or drugs independently of the defibrillation circuit.

As seen in FIG. 6, the base unit 54 includes a DC power supply 60 which is the source of energy for imparting defibrillation and drug delivery. A control circuit 62 may be hard-wired to provide both defibrillation energy and drug delivery energy at specified times and sequences once the operator activates the apparatus, for example, by pushing an “on” button 64. A separate button or switch 65 may be provided to enable the operator to initiate drug delivery. For example, in the instructions provided with the apparatus, the operator may be told to push the drug delivery button 65 after delivery of a defibrillation shock. In the absence of a drug delivery button, the apparatus may include programming or circuitry that automatically initiates drug delivery through the drug delivery circuit.

FIG. 7 illustrates an embodiment in which the defibrillation apparatus 66 includes a base unit 68, two defibrillation electrodes 70 and 72, and a drug delivery electrode 74. In appearance, the electrode 74 can resemble the defibrillation electrodes in having a non-conductive substrate, a conductive layer, and a gel layer, with the distinction being that the gel layer will include a therapeutic agent. Also, the amount of electric energy supplied to the drug delivery electrode will be of a smaller magnitude; voltages, pulse rates and durations can be selected to optimize delivery of a particular drug. As with the other electrodes, the drug delivery electrode 74 is attached to the skin of the subject for whom a defibrillation procedure is being initiated.

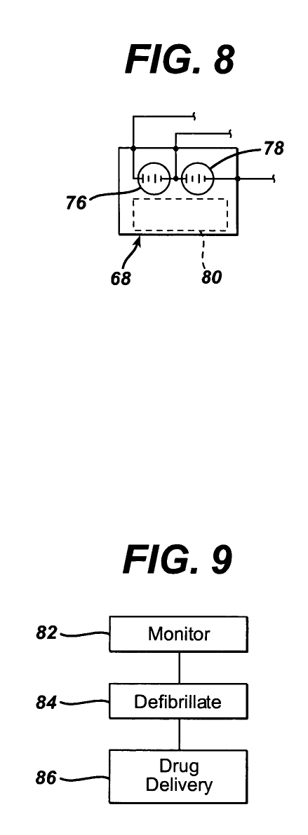

In the embodiment of FIG. 7, the drug delivery electrode 74 may be coupled to a separate power source. Referring to FIG. 8, the base unit 68 may include a first power supply 76 for providing electric energy to the defibrillation electrodes and a second power supply 78 for providing electric energy to the drug delivery electrode 74 at levels and for times sufficient to impart drug delivery. The power supply 78 may be connected between the drug delivery electrode 74 and one of the defibrillation electrodes as shown in FIG. 8.

The control circuit 80 can be programmed or wired to switch the different power supplies on and off at preferred times and durations. Also, the control circuit may include means for adjusting the power output to the electrodes depending on subject-dependent parameters.

Operation of the defibrillator to accomplish both the defibrillation function and the drug delivery function can either be automatic, manual, or a combination of both. In the various embodiments described herein, the control circuit may include a microprocessor or any other integrated circuit means which includes or is coupled to a memory for storing electrical parameters for operation of the apparatus in a defibrillation mode and a drug delivery mode. Moreover, multiple parameters can be stored, corresponding to multiple types of drugs, for use in the drug delivery mode, and multiple parameters can be stored for operation at different levels in the defibrillation mode. The selection of electrical parameters for drug delivery is dependent on the type and dosage of drug as well as the desired rate of delivery. Thus, these values can be stored in a look-up table as part of the programming of the microprocessor or permanently stored in ROM (read-only memory).

It is further possible to monitor the heart condition of the patient through an additional sensor and electrical lead or by using the electrodes and their electrical leads so that the control circuit can indicate to the user the times to defibrillate or to deliver medication. Preferably, the drugs are incorporated into the electrodes and are electrically isolated so that each can be delivered separately, if multiple drugs are provided, and if multiple doses are used. In some instances only drug delivery may be called for. At other times there may not be time or the desirability for drug delivery and the defibrillation mode is immediately selected. After defibrillation, drug delivery may then be selected manually or automatically. In any event, selection of the drug delivery mode can be manual, meaning by user selection, or automatic, meaning following execution of a software routine, based simply on timing or based on a comparison of sensed heart parameters to stored heart parameters.

A simple flow chart indicating how to program the unit is shown in FIG. 9. The first step 82 is “monitor,” in which sensors connected to a person who might be experiencing a cardiac event produce signals indicative of the condition which are fed into a memory device, such as a RAM or other suitable device, for comparison to stored values. As a result of this comparison a visual display may prompt the operator to initiate defibrillation by actuating an “on” switch. This is indicated by the step 84 for “defibrillate,” in which a defibrillator voltage is applied to the electrodes for a predetermined time and at a predetermined level. Defibrillation may occur by automatic program execution, thus eliminating the need for an operator to push the “on” button. Drug delivery may be desirable prior to providing a defibrillation shock.

Following defibrillation, the program may provide a drug delivery step 86 in which the drug delivery electrode is powered to impart transdermal drug delivery. The base unit may be provided with a display which, after a predetermined time after defibrillation, tells the operator to turn on the drug delivery electrode. This would require a second button or switch on the base unit, such as button 65 shown in FIG. 6. When the button 65 is pushed, the control circuit delivers a voltage to the drug delivery electrode for a predetermined time and at a predetermined energy level. Optionally, the control circuit may include a timer so that drug delivery is initiated automatically after defibrillation, thus minimizing operator interaction.

“Monitoring” can occur manually, such as by a user checking the pulse, checking breathing, etc., to determine the condition of a person who might be experiencing a cardiac event; in the event of manual checking, the software routine need not include a monitoring step. If monitoring is done manually, the “defibrillate” step is done manually by user manipulation of a switch. If drug delivery mode is selected, either manually or automatically, the system can be programmed to automatically select a drug or multiple drugs and the dosage, if the apparatus is provided with multiple, electrically isolated drug patches or drug delivery electrodes (which may be incorporated on a single electrode).

The program sets the electrical parameters, optionally to provide for electroporation to reduce the skin barrier to transdermal medication flux, prior to initiating electro-osmosis. Thus, the program can establish the electric potentials required to provide electroporation prior to drug delivery and electro-osmosis during drug delivery. These potentials provide an electro-motive force of sufficient strength to transport drugs into the bloodstream of the person undergoing a cardiac event at a desired dosage and rate. When the electrodes are coupled to the power source, preferably a DC power supply, the program or circuitry of the apparatus provide s voltage and/or current levels sufficient to accomplish electroporation (optionally) followed by the delivery dosage and rate.

In the defibrillation mode, electrical parameters are preferably set automatically and the electrodes are coupled to the power supply to deliver the defibrillation shock. The general operation of the defibrillator in this mode is well understood from various patent to Heartstream, In., including the aforementioned U.S. Pat. Nos. 5,607,454 and 5,466,244, which are hereby incorporated by reference.

In the simplest implementation of the present invention, the drugs are prepackaged in the electrodes, and no selection process is required; the user simply attaches the electrodes to the person undergoing a cardiac event. Usually, no drug delivery is desired before defibrillation, although the device may be programmed to do so. This is true only because administering drugs delays defibrillation. It may be preferable to deliver drugs first via automation if defibrillation is not delayed. Preferably, immediately after defibrillation, a DC current of sufficient duration and magnitude is supplied to the drug delivery electrode or portion of an electrode to cause release of the drugs and their transfer across the skin interface and into the circulatory system.

The circuitry described above may be designed or controlled by a programmed microprocessor to deliver a voltage at levels and for times sufficient to deliver drugs from one or more transdermal patches. The patches may be separate from the shock delivering electrodes of the defibrillator, or may be coincident with the defibrillation electrodes. In any event, the drug delivery voltages can be pulsed at high or low voltages. For high voltages the voltage values can range from 30 to 2500 volts, for durations of between 0.5 milliseconds and 5 seconds. The voltage is delivered through electrode patches that carry the drug for the purpose of electrically enhancing the transdermal administration of the medication, and more specifically for electroporation of the stratum corneum.

For lower voltages, the voltages are pulsed between 0 and 50 volts for durations between 0.1 second and thirty minutes. The voltage is delivered through electrode patches that carry the drug for the purpose of electrically enhancing the transdermal administration of the medication, and more specifically, for iontophoretic assistance of transport for ionic medications. Other voltages and durations, as well as other transport phenomena, can be used.

Reference herein to a “gel” is a reference to a preferred carrier for the drug, in that AED's are currently available that use gel adhesive layers to attach the defibrillation pads or electrodes to the subject. The term “carrier” is used to indicate that the therapeutic agent or drug is carrier by another substance, which could be the material that forms the gel layer of known defibrillation electrodes, or it could encompass other media, such as paste or creams which have little or no adhesive characteristic. Although it is conceivable that the drug could be applied to the skin separately from the electrode structure, this might require more operator intervention than is desired, and thus such drug applications would be less preferred.

1. An electrode for attachment to the skin of a subject during an external defibrillation procedure, comprising:
a conductive member having an outer surface; and
a therapeutic agent disposed in surface contact with the skin of a subject undergoing the defibrillation procedure and in electrical contact with the conductive member, whereby transdermal transport of the therapeutic agent to the subject is enhanced by application of electrical energy to the conductive member.

2. An electrode according to claim 1, wherein the therapeutic agent is selected from the group consisting of epinephrine, adenosine, bretylium, atropine sulfate and lidocaine.

3. An electrode according to claim 1, further comprising a gel layer covering at least a portion of the outer surface of the conductor, wherein the therapeutic agent is disposed in the gel layer.

4. An electrode according to claim 1, wherein the conductive member receives electrical energy at a level sufficient to induce at least one of electroporation and electromotion.

5. An electrode for attachment to the skin of a subject during an external defibrillation procedure, comprising:
a first conductive member having an outer surface;
a second conductive member having an outer surface and being electrically isolated from the first conductive member;
means for connecting the first conductive member to the subject;
means for connecting the second conductive member to the subject; and
a therapeutic agent in surface contact with the skin of the subject undergoing a defibrillation procedure and in electrical contact with the second conductive member, whereby transdernial transport of the therapeutic agent is enhanced by application of electrical energy to the second electrode.

6. An electrode according to claim 5, wherein the first and second conductive members are carried by a single non-conducive substrate.

7. An electrode according to claim 6, wherein the first and second conductive members are substantially coplanar.

8. An electrode according to claim 5, wherein the therapeutic agent is a drug selected from the group consisting of epinephrine and lidocaine.

9. An electrode according to claim 5, wherein the means for attaching the first and second conductive members includes, respectively, first and second gel layers which are electrically conductive, each having an inner surface connected respectively to the first and second conductive members.

10. An electrode according to claim 5, wherein the second conductive member receives electrical energy at a level sufficient to induce at least one of electroporation and electromotion.

11. An external defibrillation apparatus, comprising:
a power supply;
a control circuit connected to the power supply;
first and second electrodes electrically connectable to the power supply through the control circuit, and being connectable to the skin of a subject undergoing a defibrillation operation; and
a therapeutic agent in electrical contact with at least one of the first and second electrodes, the at least one electrode being electrically powered at a level sufficient to enhance transdermal transport of the therapeutic agent to the subject.

12. A defibrillation apparatus according to claim 11, wherein each electrode includes a conductive member having first and second opposite side surfaces, and a non-conductive backing connected to the first surface of the conductive member.

13. An defibrillation apparatus according to claim 11, wherein the first and second electrodes includes a gel layer, and therapeutic agent is carried by the gel layer of at least one of the electrodes.

14. A defibrillation apparatus according to claim 11, wherein the first and second conductive member receive electrical energy at a level sufficient to induce at least one of electroporation and electromotion.

15. A defibrillation apparatus according to claim 11, wherein the therapeutic agent is a drug selected from the group consisting of epinephrine and lidocaine.

16. A defibrillation apparatus according to claim 12, wherein the therapeutic agent is carried by an electrically conductive gel layer connected to one of the first and second conductive members.

17. A defibrillation apparatus according to claim 11, wherein the power supply delivers a voltage to the first and second electrodes in a range of about 30 to 2,500 volts for a time between about 0.5 milliseconds and 5 seconds, the voltage being sufficient to impart transdermal delivery of the drug and to deliver a defibrillation shock to the patient.

18. A defibrillation apparatus according to claim 11, wherein the power supply delivers a voltage to the electrodes in a range of about 0 to 40 volts for a time between about 0.1 seconds and 30 minutes, the voltage being sufficient to enhance the transdermal delivery of the drug via electromotive force.

19-22. (canceled)

23. An external defibrillation apparatus comprising:
a base unit including a power supply;
a first defibrillation electrode connectable to the power supply;
a second defibrillation electrode connect able to the power supply;
a drug delivery electrode connectable to the power supply; and
a control circuit for selectively connecting the power supply to the first, second and third electrodes to deliver electric energy at a level sufficient to defibrillate a subject and to impart transdermal delivery of a drug to the subject.

24. A defibrillation apparatus according to claim 23, wherein the power supply includes a first power supply connected between the first and second defibrillation electrodes, and a second power supply connected between one of the first and second defibrillation electrodes and the drug delivery electrode.
